(19) United States
(12) Patent Application Publication
(10) Pub. No.: US 2006/0210492 A1
(43) Pub. Date: Sep. 21, 2006

(54) Use of rifaximin for treatment and prevention of periodontal conditions

(76) Inventor: Robert E. Kodsi, Brooklyn, NY (US)

Correspondence Address: Dara L. Onofrio, Esq.;c/o ONOFRIO LAW, Suite 1600, 1133 Broadway, New York, NY, 10010, US

(21) Appl. No.: 11/365,431

(22) Filed: Mar. 1, 2006

Related U.S. Application Data
(60) Provisional application No. 60/657,783, filed on Mar. 2, 2005.

(51) Int. Cl. A61K 8/49; A61K 31/496

(52) U.S. Cl. 424/49; 514/252.13

(57) ABSTRACT

An oral preparation used for treatment and prevention of bacterial periodontal conditions consisting of a non-systemic antibiotic; and a method of treatment of periodontal conditions by orally administering to a subject in need of such treatment a composition containing a therapeutically effective amount of rifaximin.

This application claims the benefit of U.S. provisional application No. 60/657,783 filed Mar. 2, 2005, which is incorporated herein in its entirety by reference.

### FIELD OF THE INVENTION

This invention relates to use of rifaximin compositions in the prevention and treatment of periodontal conditions. In particular, the compositions are used for prevention and treatment of periodontal conditions caused by bacteria such as plaque, tooth decay and gingivitis.

### BACKGROUND OF THE INVENTION

In general, rifaximin is well known as a non-systemic antibiotic (<0.4%) characterized by activity against a broad spectrum of enteric bacterial pathogens and the delivery of high concentrations of antibiotic to the gastrointestinal tract.

The antibiotic rifaximin was discovered in 1980 and originally patented in Italy as IT Patent 1154655 granted on Jan. 21, 1987. The related U.S. Pat. No. 4,341,785 to Marchi et al. discloses imidazo-rifamicyn derivatives having antibacterial utility, and the related process for preparing it. The '785 Patent also discloses a pharmaceutical antibacterial composition and a method of using it to treat antibacterial diseases of the gastrointestinal tract. A further patent, U.S. Pat. No. 4,557,866 to Cannata et al. discloses a process for the synthesis of pyrido-imidazo rifamycins. The process is described as an improvement over the '785 Patent to Marchi in that the later process provides unsatisfactory yields from an industrial point of view.

Rifaximin is essentially a non-absorbable semi-synthetic antibiotic, related to rifamycin. The antimicrobial spectrum (in vitro) includes most gram-positive and gram-negative bacteria; and both aerobes and anaerobes.

It presents low risk for drug interactions (no effect on drugs metabolized by cytochrome p450 enzyme system) and about the same adverse properties as compared to a placebo. When ingested in tablet or pill form rifaximin is concentrated in the gastrointestinal tract and primarily excreted unchanged in the feces. It binds to the beta subunit of bacterial DNA-dependent RNA polymerase, which inhibits bacterial RNA synthesis. In contrast with other antibiotics, resistance to rifaximin is not plasmid-mediated but utilizes a chromosomal one-step alteration in the DNA-dependent RNA polymerase. In subjects using rifaximin no relevant resistance has been observed. Further, mutant resistant bacteria showed reduced viability and there is no systemic cross resistance for rifampin.

Since rifaximin is practically insoluble in water and is non absorbed (<0.4%) after oral administration, it can be used to treat localized diseases of the gastrointestinal tract. Rifaximin products specific for enteric pathogens of the gastrointestinal tract are presently commercially marketed under various trade names—NORMIX® available from Alfa Wassermann S.p.A., Bologna, Italy; XIFAXAN® available from Salix Pharmaceuticals, Raleigh, N.C.; REDACTIV® available from GlaxoSmithKline and FLONORM® from Schering-Plough. Since the solubility of rifaximin in water is approximately 1 μgmL³ the drug is virtually undissolved when traveling through the GI tract. The relative insolubility of rifaximin is thought to influence bacterial susceptibility and subsequent eradication due to the invasive nature of some enteric pathogens (e.g. Salmonella and Campylobacter). The relative insolubility of rifaximin also leads to its negligible systemic absorption. Rifaximin has been known to be effective for treating infections that are localized to the gut and is not known to be suitable for treating systemic infections caused by invasive organisms.

Rifaximin has been marketed in Italy since 1985 under the trademark NORMIX® for treating acute and chronic intestinal infections from gram-positive and gram-negative bacteria and as adjuvant in the therapy of the hyperammonoaemia. At present NORMIX® is marketed in the shape of pharmaceutical compositions, orally administrable, made by tablets or by granulates containing suitable pharmaceutically acceptable excipients together with rifaximin, but also other pharmaceutical forms orally administrable like capsules, sugar coated tablets and syrups can be used.

Xifaxan® is marketed in the United States and Canada and includes rifaximin as the active ingredient. The formulation is used in the treatment of travelers' diarrhea caused by the noninvasive strains of Escherichia coli. Xifaxan® is a non-absorbable antiobiotic for gastrointestinal infections. Dr. Herbert DuPont, director of the Center for Infectious Diseases at the University of Texas, School of Public Health, developed the drug for treatment of travelers' diarrhea. DuPont said “the drug is unique in that it remains in the gastrointestinal tract, compared with powerful antibiotics like Cipro that disperse throughout the body. This means the drug is less likely to breed resistant bacteria.” He said the antibiotic proved 85% effective in protecting US students who participated in a two-week study trip to Mexico, versus just 49% who didn't become sick on non-medicinal placebos. The drug has been found to have no significant side effects.

Products similar to NORMIX® and Xifaxan® are marketed in Mexico under the tradenames REDACTIV® and FLONORM®.

Other uses of rifaximin are disclosed in the following patents:

U.S. Pat. No. 5,886,002 to Ferrieri et al. describes use of rifaximin compositions in the treatment of diarrhea from cryptosporidiosis.

U.S. Pat. No. 5,352,679 to Ferrieri et al. describes use of rifaximin (INN) in formulations for treatment of gastric dyspepsia caused by Helicobacter pylori bacteria.

U.S. Pat. Nos. 5,314,904 and 6,140,355 both to Egidio et al. disclose compositions containing rifaximin for treatment of vaginal infections.

Known therapeutic uses of rifaximin, administered in a tablet form, include Clostridum difficile-associated diarrhea, Crohn's disease, Diverticular disease, Hepatic encephalopathy, Helicobacter pylori eradication, infectious diarrhea, irritable bowel syndrome, pouchitis, prophylaxis for GI surgery, small bowel overgrowth, traveler's diarrhea and ulcerative colitis. These therapies are directed to pediatric, adult and elderly subjects.

At present rifaximin has been studied and marketed only for the treatment of some kinds of bacterial infections located in the gastro-intestinal and reproductive tract, however treatment for bacterial aggregation in the oral cavity has never been investigated.

Plaque, gingivitis and tooth decay are endemic problems in the world, even with advances in brushing, fluoride treatments, flossing and better dental care. Seventy five percent of Americans over the age of 35 have some form of gum disease with thirty percent of the population being genetically susceptible (6 times the average risk) for developing gum disease.

U.S. Pat. No. 5,082,653 to Pan et al. discloses alcohol compositions bound to an antibiotic to provide anti-plaque and anti-gingivitis activity. The compositions provided a synergistic combination of a morpholinoamino alcohol, such as 3-(4-propylheptyl)-4-(2-hydroxyethyl)morpholine and an antibiotic such as the aminoglycosides, amphenicols, ansamycins, carbapenems, cephalosporins, cephamycins, monobactams, oxacephems, penicillins, lincosamides, macrolides, polypeptides and tetracyclines. Constant use of these formulations which include an antibiotic component leads to antibiotic resistance and to the destruction of good bacteria.

Antibiotics, such as rifaximin, that are non-absorbed by the body, have not been used to treat or to prevent periodontal conditions, in particular conditions caused by bacteria that lead to plaque, tooth decay and gingivitis. The present invention provides advantage in doing so such that there are no systemic side effects. The invention oral preparations which contain rifaximin directly target the cause of plaque and tooth decay without causing systemic harm to the person.

Accordingly the present invention is directed to use of rifaximin in toothpaste and in mouthwash to prevent periodontal conditions such as bacterial plaque, tooth decay and gingivitis. The compositions can also be impregnated in floss or applied directly to the gums via a dental rubber tip instrument to prevent periodontal conditions as previously described. Rifaximin can also be placed in disks, gels, glue or actisite and can be applied or injected into the sulcus or any portion of the oral cavity.

### SUMMARY OF THE INVENTION

The present invention provides a method of treatment of periodontal conditions which consists of orally administering to a subject in need of such treatment a composition containing a therapeutically effective amount of rifaximin.

The invention method and treatments are effective against periodontal conditions caused by bacteria. They also are believed to be effective against periodontal conditions which are caused by protozoa, mycobacterium, RNA dependent viruses, reverse transcriptase dependent viruses and any other infections etiology that utilizes RNA.

The composition is preferably a pharmaceutical composition.

Electrolytic reduction of rifaximin produces a slightly different structure referred to as rifaximin OR (open ring). As used in the specification herein, when the term rifaximin is used it is intended to also include the rifaximin OR structure unless otherwise stated. It is believed that the two molecules are similar in structure but have different chemical properties in solution.

In general, in the formulations of the invention, the therapeutically effective amount of rifaximin preferably delivers a dosage to achieve a concentration of up to 10,000 or more μg/ml per application. It is believed that dosages in the concentration range between 1-1000 μg/ml per application would also be effective. The rifaximin preparations work on the surfaces to which they are applied with essentially no absorption into the tissue itself. The duration of treatments with the invention formulations can be from one to three times per day to once a month depending on the individual and the desired outcome.

Rifaximin as a powder or solid granular form is incorporated into a tooth paste. Since the rifaximin is essentially non-reactive with other compounds, it can be incorporated into any commercially available tooth paste formulation without losing its efficacy. Additional compounds such as fluoride, chlorohexidine, triclosan and other similar compounds can be added to the tooth paste formulations.

Rifaximin as a powder or solid granular form is incorporated into a mouthwash. Since the rifaximin is essentially non-reactive, it can be incorporated into any commercially available mouthwash formulation without losing its efficacy. Additional compounds such as fluoride, chlorohexidine, triclosan and other similar compounds can be added to the mouthwash formulations.

Rifaximin as a powder or solid granular form is incorporated into a paste or solution which is applied to the gums of a subject via a dental rubber tip instrument. Since the rifaximin essentially is non-reactive, it can be incorporated into any aqueous or non-aqueous solutions without losing its efficacy. Additional compounds such as fluoride, chlorohexidine, triclosan and other similar compounds can be added to these formulations.

Dental floss is impregnated with a rifaximin solution and used by the subject in a conventional manner. Since the rifaximin essentially is non-reactive, it can be incorporated into any aqueous or non-aqueous solutions without losing its efficacy. Additional compounds such as fluoride, chlorohexidine, triclosan and other similar compounds can be added to these formulations. The dental floss may be with or without wax as a component.

Any oral preparation of rifaximin that will target the source of bacterial periodontal conditions that is applied in the oral cavity or over the teeth and the gums is encompassed by the scope of the invention.

Although this disclosure is directed to the preferred use of rifaximin, it is also within the scope of the invention that any non-systemic antibiotic will have the same effect and is included herein.

Other objects, features and advantages of the present invention will be apparent when the detailed description of the preferred embodiments of the invention are considered with reference to the drawings, which should be construed in an illustrative and not limiting sense as follows:

### BRIEF DESCRIPTION OF THE DRAWINGS

FIG. 1 is the chemical structure of rifaximin.

### DETAILED DESCRIPTION OF THE INVENTION

In general, periodontal disease is an inflammation or degeneration of tissues that surround and support the teeth: gingiva, alveolar bone, periodontal ligament, and cementum. Periodontal disease most commonly begins as gingivitis and progresses to periodontitis. If the severity of the disease is disproportionate to the amount of plaque and calculus, systemic disease may be present.

Gingivitis is an inflammation of the gingival, characterized by swelling, redness, change of normal contours, watery exudates, and bleeding. Swelling deepens the crevice between the gingival and the teeth, and gingival pockets form. Gingivitis is common and may be acute, chronic, or recurrent. The most frequent single cause is poor hygiene, characterized by bacterial plaque (microbial colonies tenaciously attached to the tooth surfaces). Other local factors such as malocclusion, dental calculus (calcified plaque called tartar), food impaction, faulty dental restorations, and mouth breathing play important secondary roles Current treatments for the disease is to control or correct both plaque and systemic factors;

Periodontitis is a progression of gingivitis to the point that loss of supporting bone has begun. It is the primary cause of tooth loss in adults.

Tooth decay is a location on a tooth where its mineral content has been dissolved away and a hole has formed (demineralization). Demineralization of a tooth is caused by acids, acids that are created by certain types of bacteria that live in our mouths. The bacteria consume the food—namely sugars and the waste products that these bacteria create from having consumed these sugars are acids which cause tooth decay. To prevent tooth decay one must not allow the bacteria that are present in the mouth to form organized colonies on the surface of the teeth—called dental plaque.

Current research demonstrates that contolling plaque is important in the control of tooth decay and gum disease. Plaque is a clear, sticky deposit of bacteria that adhere to the surface of teeth and gum tissue. Plaque contains a variety of different types of bacteria. As a result, certain types of plaque are associated with dental decay, others with calculus formation, and others with the inflammatory response of the gums (gingivitis).

Plaque begins forming on the teeth as little as four hours after brushing. The rate at which plaque forms and the location in which it develops can vary between individuals and even between different teeth in the same mouth. One of the prime areas in which plaque accumulates is at the gingival margin and sulcus where the tooth meets the gum.

Plaque which is not removed by brushing and flossing can harden into calculus (also called tartar). Calculus is plaque that has mineralized, forming a tough, crusty deposit. The deposits can form above (supragingival) and below (subgingival) the gum line. Calculus deposits are a significant contributing factor in periodontal disease because it is always covered by a layer of nonmineralized plaque. The calculus keeps the plaque close to the gingival tissue and makes it more difficult to remove the plaque bacteria.

There are basically two types of Plaque:

1. Attached Plaque (AP) found above the gum line is by far the least toxic and

2. Loosely Attached Plaque (LAP) found below the gum lines contains up to 60 times the concentration of toxins seen in AP. Spirochetes are corkscrew shaped bacteria found in LAP and are the center of periodontal disease activity. LAP is easily diluted and reduced with irrigation.

The present invention, in contrast to known methods and treatments, provides an oral preparation used for treatment and prevention of bacterial periodontal conditions consisting of a non-systemic antibiotic,

More particularly, it provides a method of treatment of periodontal conditions caused by bacteria, which consists of orally administering to a subject in need of such treatment a composition containing a therapeutically effective amount of rifaximin.

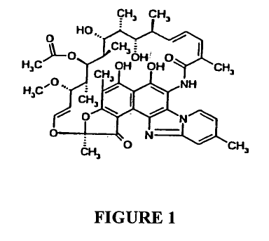

Rifaximin is a semi-synthetic, non-systemic antibiotic. The chemical name for rifaximin is (2S,16Z,18E,20S,21S,22R,23R,24R,25S,26S,27S,28E)-5,6,21,23,25-pentahydroxy-27-methoxy-2,4,11,16,20,22,24,26-octamethyl-2,7-(epoxypentadeca-[1,11,13]trienimino)benzofuro[4,5-e]pyrido[1,2-a]-benzimidazole-1,15(2H)-dione,25-acetate. The empirical formula is C43H51N3O11 and its molecular weight is 785.9. The chemical structure is shown in FIG. 1.

Over 500 types of bacteria have been identified in the human mouth. Typical strains include Actinobacillus actinomycetumcomitans, Porphyromanas gingivalis, Bacteriodes forcythus, Treponema denticola, Prevotella intermedia, and Eubacterium nodatum. The invention preparations and method of treatment help control these bacteria to prevent periodontal conditions such as plaque, tooth decay and gingivitis.

It is also contemplated within the invention, that the rifaximin treatments would be effective against protozoa, mycobacterium, RNA dependent viruses, reverse transcriptase dependent viruses and any other infections etiology that utilizes RNA.

Rifaximin is essentially non-reactive and can be incorporated into various solutions without losing its efficacy. The solid rifaximin material is provided in powder or as particulate granules does not dissolve but disperses upon placement in solution.

Compositions containing a therapeutically effective amount of rifaximin are orally administered to a subject in need of such treatment. The composition is preferably a pharmaceutical composition and contains a therapeutically effective amount of rifaximin which preferably delivers a dosage to achieve a concentration of up to 10,000 or more μg/ml per application. It is believed that dosages in the concentration range between 1-1000 μg/ml per application would also be effective. The duration of treatments with the invention formulations can be from one to three times per day to once a month depending on the individual and the desired outcome

The invention includes use of rifaximin in toothpaste and mouthwash compositions, which may further include fluoride, chlorohexidine, triclosan and other similar compounds. Rifaximin may also be impregnated in a dental floss or incorporated into a paste or solution to be applied to the gums or a subject via a dental rubber tip instrument. The invention also includes any oral preparation of rifaximin that is applied within the oral cavity or over the teeth and gums. In all composition embodiments of the invention are used to treat and prevent periodontal conditions caused by bacteria, including plaque, tooth decay and gingivitis.

Additionally, rifaximin can be placed in disks, gels, glue or actisite and can be applied or injected into the sulcus or any portion of the oral cavity. For example, a rifaximin containing gel can be injected into the sulcus, which is the pocket between the tooth and gums. A fabric filament such as actisite can be impregnated with the rifaximin composition and placed into the sulcus for a period between 1 to 14 days depending on condition being treated. A slowly dissolving “chip” or disk containing the rifaximin composition can be placed into the sulcus. The rifaximin composition can be incorporated into glue which can be used in the oral cavity. Finally, a material to be used as a membrane to cover a bone graft during oral surgery can be impregnated with the rifaximin composition to help prevent infection. The material can be synthetic, semi-synthetic or natural. Preferred material used is gortex. The composition can be impregnated into a material which can be temporary or permanent and can be grafted into or placed in the oral cavity.

The compositions can be used on a regular or intermittent basis, ranging from one to three times a day to once a month, or other intervals, depending on the individual and the desired results.

The present invention will be illustrated in more detail by the following examples without limiting the scope of the invention in any way.

### EXAMPLE 1

Approximately 200 mg of solid particulate rifaximin was incorporated into a commercial mouth wash product. The mixture was shaken and the rifaximin dispersed in the solution. A 30 cc portion of this combined solution was used by a subject to rinse their oral cavity. The solution was swirled around the oral cavity for seconds and then expelled by the subject. This procedure was followed twice a day for a three day period.

### EXAMPLE 2

Approximately 200 mg of solid particulate rifaximin was incorporated into a commercial tooth paste product. The rifaximin dispersed in the toothpaste. A small bead of the invention tooth paste was applied to a tooth brush and used by a subject to brush their teeth and gums. This procedure was followed twice a day for a three day period.

The invention now being fully described, it will be apparent to one of ordinary skill in the art that many changes and modifications can be made thereto without departing from the spirit or scope of the invention as set forth herein.

What is claimed is:

1. A method of treatment of periodontal conditions, which consists of orally administering to a subject in need of such treatment a composition containing a therapeutically effective amount of rifaximin.

2. A method according to claim 1 wherein the periodontal conditions are caused by bacteria.

3. A method according to claim 1, wherein the periodontal conditions are caused by protozoa, mycobacterium, RNA dependent viruses, reverse transcriptase dependent viruses and any other infections etiology that utilizes RNA.

4. A method according to claim 1 wherein said composition is a pharmaceutical composition.

5. A method according to claim 1 wherein said therapeutically effective amount of rifaximin delivers a dosage to achieve a concentration of up to 10,000 or more μg/ml per application.

6. A method according to claim 1 wherein said therapeutically effective amount of rifaximin delivers a dosage to achieve a concentration between 1-1000 μg/ml a day.

7. A method according to claim 1 wherein said composition is a tooth paste.

8. A method according to claim 1 wherein said composition is a mouthwash.

9. A method according to claim 1 wherein said composition is applied to the subjects gums via a dental rubber tip instrument.

10. A method according to claim 1 wherein said composition is impregnated in floss.

11. A method according to claim 1 wherein said composition further contains fluoride, chlorohexidine, triclosan or other similar compounds.

12. A method according to claim 1 wherein said composition is a disk, gel, or glue.

13. A method according to claim 1 wherein said composition is a gel which is injected into the sulcus.

14. A method according to claim 1 wherein said composition is impregnated in a fabric filament and placed into the sulcus.

15. A method according to claim 1 wherein said composition is a glue used in the oral cavity.

16. A method according to claim 1 wherein said composition is a slowly dissolving chip or disk which is placed in the sulcus.

17. A method according to claim 1 wherein said composition is impregnated into a material used as a membrane to cover a bone graft during oral surgery.

18. A method according to claim 1 wherein said composition is impregnated into a material which can be temporary or permanent and can be grafted into or placed in the oral cavity.

19. An oral preparation used for treatment and prevention of bacterial periodontal conditions consisting of a non-systemic antibiotic.

20. The oral preparation according to claim 19 wherein said non-systemic antibiotic is rifaximin.

21. The oral preparation according to claim 19 wherein said non-systemic antibiotic is delivered to a subject in a dosage to achieve a concentration of up to 10,000 or more μg/ml per application.
